(19) United States
(12) Patent Application Publication
(10) Pub. No.: US 2020/0206039 A1
(43) Pub. Date: Jul. 2, 2020

(54) Toilet Training Undergarment Disposable Insert Liner

(71) Applicant: Elizabeth Theresa McLain, Kenilworth, NJ (US)

(72) Inventor: Elizabeth Theresa McLain, Kenilworth, NJ (US)

(21) Appl. No.: 16/112,671

(22) Filed: Aug. 25, 2018

(51) Int. Cl. A61F 13/47; A61F 13/511; A61F 13/512

(52) U.S. Cl. CPC A61F 13/47; A61F 13/5116; A61F 2013/51023; A61F 2013/4708; A61F 13/5123

(57) ABSTRACT

The present invention describes an undergarment pantiliner which is worn with a child's underwear, to aid in toilet training while protecting undergarments from soiling. The invention is directed to in particular the situation in which the child has mastered urination but continues to have challenges with controlling bowel movements. The pantiliner is made up of sheets of non-woven disposable fabric and other materials which protect the wearer's underwear from stains and/or exudates. It is meant to absorb a little wetness but let most fluid flow through while retaining excrement and exudate. Use of the item aids the child's self-confidence to believe he can transition to be fully toilet-trained by supporting his sense of independence.

### CROSS-REFERENCE TO RELATED APPLICATIONS

This application claims the benefit of U.S. Provisional Application No. 62/553,803, filed on Sep. 2, 2018

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field of absorbent articles and sanitary napkins. During toilet training, some children become partially toilet trained before they become fully toilet trained. Some become trained with urination first and there is time before they would become stool trained. In some other cases, the child is trained for bowel movements first, and then urination training takes more time. The present invention deals with the first case in which the child has been trained for urination but lags in stool training. In some of these cases, the child is withholding stool which results in later soiling his underwear. At some point, 20% of children refuse to stool train[Issenman, R M, Filmer, R B, Gorski, P A. A Review of Bowel and Bladder Control Development in Children: How Gastrointestinal and Urologic Conditions Relate to Problems in Toilet Training. Pediatrics. June 1999; 103(3)]. 1 to 3% of children are affected by encopresis or chronic fecal soiling at some point.

Some researchers estimate that up to 30% of children between age 2 and 10 have chronic constipation [Van den Berg M M, Benninga M A, Di Lorenzo C. Epidemiology of childhood constipation: a systematic review. American Journal of Gastroenterology. October 2006; 101(10):2401-9].

The purpose of the invention is to prevent soiling of underwear when the child is having difficulties in stool training or from being affected by encopresis or other issues causing undergarment/underwear soiling.

### SUMMARY OF THE INVENTION

The present invention is a sanitary napkin meant to prevent soiling of the user's undergarment. The present disclosure describes an undergarment pantiliner which is worn with a child's underwear, to aid in toilet training while protecting undergarments from soiling. The invention is directed to in particular the situation in which the child has mastered urination but continues to have challenges with controlling bowel movements. The pantiliner is made up of sheets of non-woven disposable fabric and other materials which protect the wearer's underwear from stains and/or exudates. It is meant to absorb a little wetness but let most fluid flow through while retaining excrement and exudate. Use of the item aids the child's self-confidence to believe he can transition to be fully toilet-trained by supporting his sense of independence.

### BRIEF DESCRIPTION OF THE DRAWINGS

FIG. 1a. Pantiliner applied to undergarment

FIG. 1b. Pantiliner general shape as applied to undergarment

FIG. 2. Cutaway View

FIG. 3a. Skin-contact sheet 4 with perforations

FIG. 3b. Skin-contact sheet 4 with perforations and embossed protrusion patterns

FIG. 4 Transfer layer (from prior art)

FIG. 5a: Thin absorbent sheet whole area (nominal)

FIG. 5b: Thin absorbent sheet whole area (gradient)

FIG. 5c: Thin absorbent sheet as sub-area within pantiliner

FIG. 5d: Pantiliner and absorbent sheet as different shape

FIG. 6a: Mesh sheet in grid pattern

FIG. 6b: Mesh sheet in grid pattern (diagonal)

FIG. 6c: Mesh sheet with variable grid sizes

FIG. 6d: Mesh sheet as sub-area within pantiliner

FIG. 6e: Mesh sheet as sub-area (reverse of FIG. 6d)

FIG. 7a: Backside Sheet with no perforations

FIG. 7b: Backside Sheet with perforations

FIG. 7c. Backside Sheet with perforations on sub-area of pantiliner area

FIG. 7d. Backside Sheet with graded perforations

FIG. 7e. Backside Sheet with graded perforations (reverse of 7d)

FIG. 8a: Alignment index pattern

FIG. 8b: Alignment with tabs

### DETAILED DESCRIPTION OF THE INVENTION

### Definitions of Terms

All cited references are incorporated herein by reference in their entireties. Citation of any reference is not an admission regarding any determination as to its availability as prior art to the claimed invention. The definitions of terms from patent U.S. Pat. No. 8,211,074 are hereby incorporated by reference. However, for the present invention, the term “pantiliner” is meant to apply for both female and male users. Unless otherwise stated, all terms referenced will be understood to apply to both genders.

### Description

The present invention describes an undergarment pantiliner that is worn with a child's underwear, in order to aid in toilet training and protect undergarments and garments against soiling. The pantiliner allows the child to wear regular child underwear with light protection in the event of an accidental leakage of liquid or solid fecal matter that could soil underwear. The liner allows the caregiver to redirect the child's toilet training. The liner aids in easier clean-up of the leakage by making cleanup faster and neater. In particular, the invention is directed towards the situation in which the child has mastered urination in the toilet but still has challenges with controlling bowel movements. The use of this invention presents several benefits to the user. It saves caregivers the time and hardship of dealing with toilet training messes in the child's underwear. It also saves the underwear from being stained and prematurely disposed of because of staining. Another benefit for the child is expression of individuality by choosing his or her own unique underwear each day and instead of the same design of pull-up diaper each day. It aids in a child's self-confidence and ability to believe he/she can be toilet-trained because the child is able to wear his or her own underwear. The use of the liner continues to support the child's sense of autonomy, self-determination, and independence. The invention would use less material than traditional diapers or pull-ups. This in turn costs less in material and means less environmental waste which is important for conscientious consumers.

The item entails a layered non-woven disposable fabric that is used as a pantiliner insert to protect the wearer's underwear from stains and/or exudates. It comprises a flat piece of layered non-woven material cut into a shape that mirrors most child underwear sizes. The piece would be cut so as to cover the area from substantially in front the user's perineum area towards the user's back expanding to cover the cloth of the underwear up until the small of the back and lateral sides (hip area).

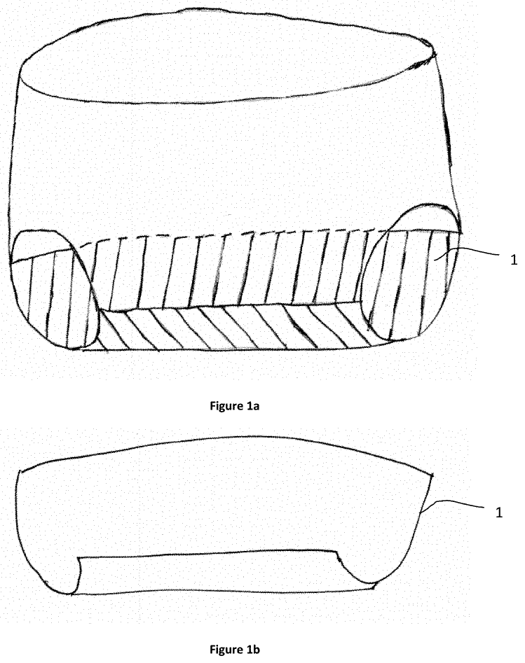

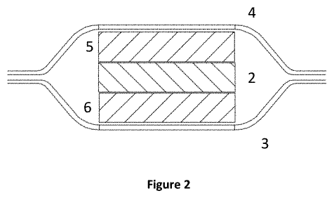

The insert liner 1 is subjected mainly to such purposes as an underwear liner, and as shown in FIG. 1, for example, has a thin absorbent member 2 which intervenes between a liquid-permeable OR impermeable backside sheet 3 and a liquid-permeable skin-contact surface sheet 4, and optionally, a hydrophilic second sheet (also referred to as a transfer sheet) 5 is disposed between the surface sheet 4 and the thin absorbent member 2. The article optionally has a liquid permeable mesh layer 6 disposed between the thin absorbent layer 2 and the backside-sheet 3. Along the perimeter of the absorbent member 2, the backside sheet 3 and the surface sheet 4 are bonded with an adhesion means, such as a hot-melt adhesive or other means.

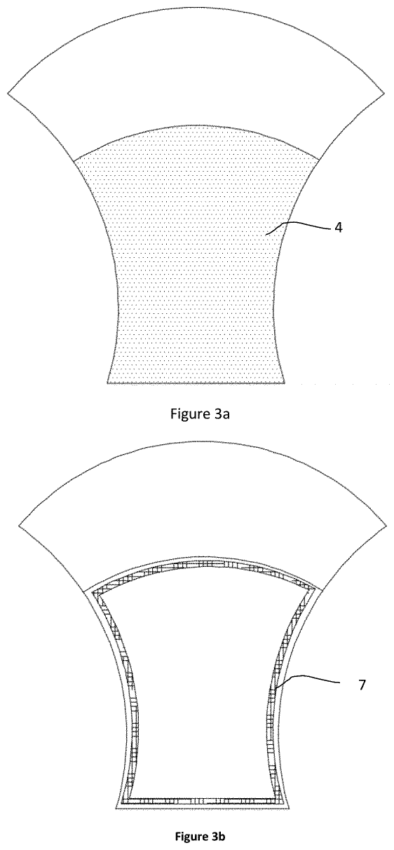

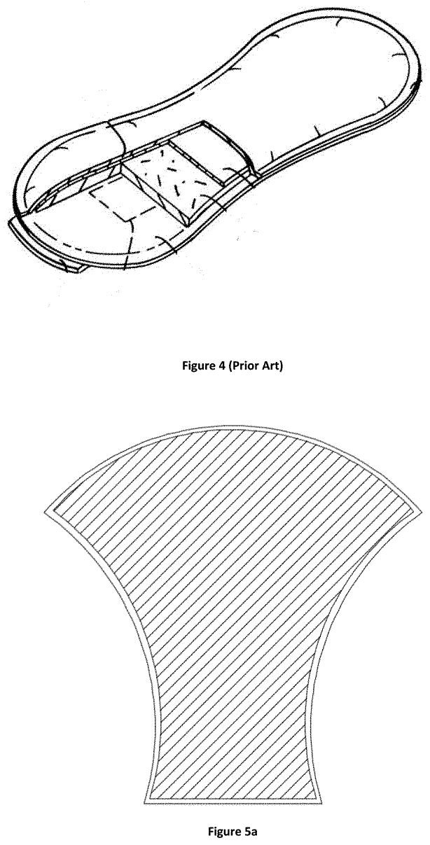

The contact surface sheet 4 may comprise layers of materials such as nonwoven fabric of olefin such as polyester, or polyamide series or may also be regenerated fibers like rayon and cupra, or natural fibers, such as cotton. The contact sheet 4 comprises two to three layers embossed in a regular pattern (shown in drawing FIG. 3a) with small perforations hole-size minimum of 0.3 mm for liquid permeability. The basis weight of the contact sheet comprising two to three layers is approximately 30 to 60 gsm. Total thickness of contact sheet 4 is preferably 5 mm or less, most preferably 2 mm thick or less. The contact surface sheet 4 and other layers may be embossed. Examples of embossing and methods are described in U.S. Pat. Nos. 8,748,692, 8,361,913 which are incorporated herein in their entirety by reference thereto for all purposes. The embossing could alternatively include addition of protrusions 7 (shown in drawing FIG. 3b) along article's border or in a pattern to retain flow of exudate within the confines of the article and to prevent soiling of user's undergarment as described in U.S. Pat. No. 7,754,940. Additionally, one could also use a “velcro” like structure contact sheet whole or in a pattern to help keep feces in place. The methods of manufacture may comprise methods as described in references U.S. Pat. Nos. 4,280,978, 4,323,068 or related methods used in the nonwoven fabric industry.

The described invention would incorporate optionally an additional transfer layer 5 (such as described in reference U.S. Pat. No. 6,206,865) disposed between skin-contact surface sheet 4 and absorbent member 2.

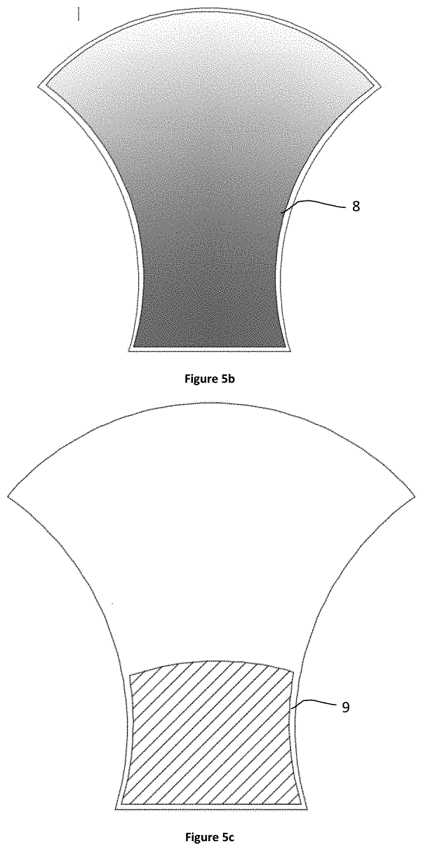

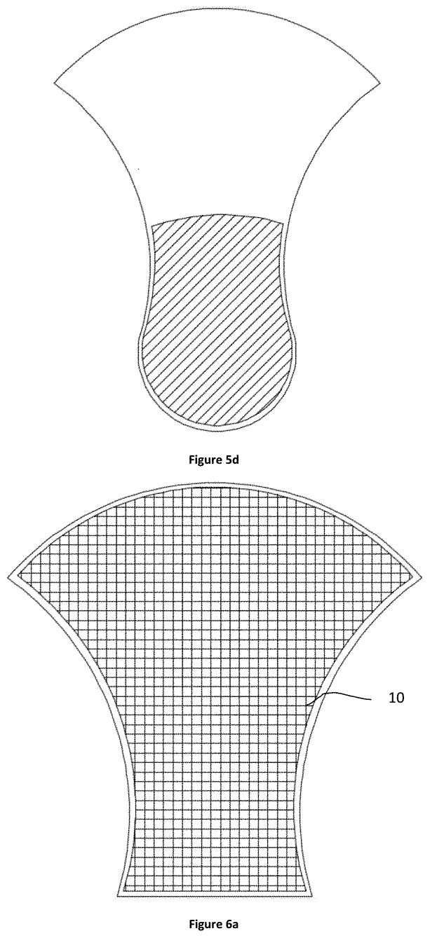

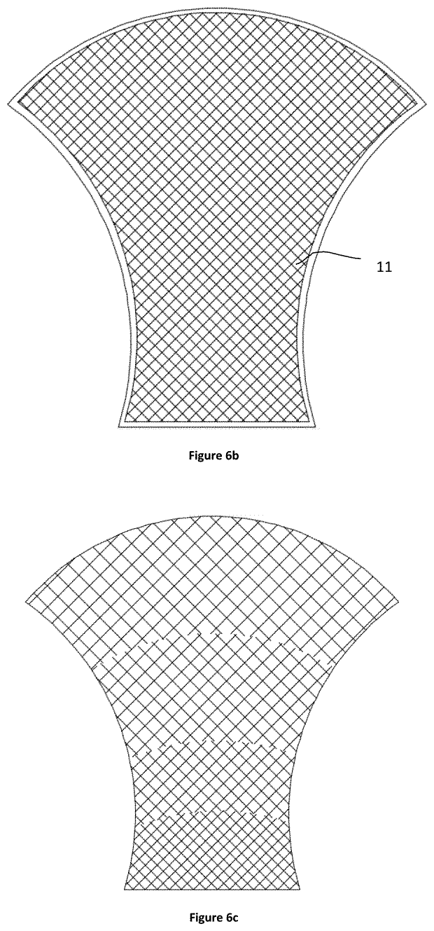

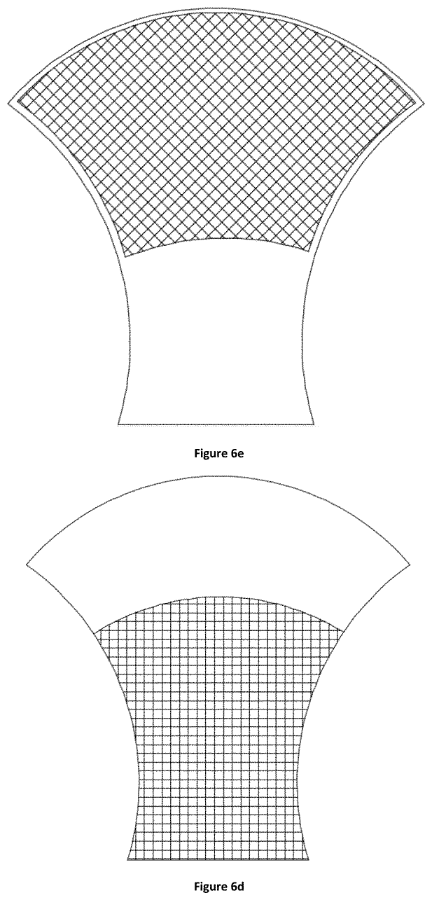

An absorbent sheet 2 is interleaved between the transfer sheet 5 and the back-side sheet 3. The absorbent sheet comprises any one of following materials or combinations thereof spread evenly to approximately 0.5 to 2 mm thick. The absorbent layer can also be unevenly spread to comprise thicker sheet approximately where urine-exudate would flow and less thick in other areas. An example gradient 8 is shown in FIG. 5b. The absorbent sheet 2 could be a sheet throughout the article assembly or preferably terminate onto a sub-area 9 of the article (shown in FIG. 5b). The material makeup of the thin absorbent sheet 2 is pulp mixed with a water-absorbing resin. Pulp materials such as cellulose from wood (soft or hardwoods) or artificial pulp made from rayon and acetate fibers. The water-absorbing resin would be materials such as acrylic acid or acrylic salt-based materials. The percentage of water-absorbing resin would be less than 10% since the main objective of the present invention is to absorb a little wetness but let most flow through while retaining excrement or exudate. Other embodiments may contain higher percentage water-absorbing resin for potential other applications or to retain more wetness. The basis weight of absorbent sheet would be approximately 20 to 100 gsm, preferably 20 to 50 gsm. Thickness of absorbent sheet would be less than 5 mm, preferably less than 2 mm thick. Manufacturing methods for making the absorbent sheet 2 would comprise those normally found in the nonwovens industry. The mesh sheet 6 entails a layered grid 10 of appropriate thermoplastic elastomer or other stretchable filaments. The materials would be blends of thermoplastic elastomers. The grid hole size may range from 1 mm to 20 mm, preferably from 5 mm to 10 mm. The grid orientation is generally diagonally disposed 11 relative to the article orientation as shown in FIG. 6d but may comprise any orientation. The grid may also be variable grid size across the sheet to change yield and strain rate based on load distribution. Alternatively, a mesh layer would be replaced by a nonwoven resilient layer. The mesh sheet comprises strands of filament material arranged in a grid-like pattern and layered into a sheet. Nonwoven layers are stretched out and interleaved with stretched out grid of filaments then when released scrunch up into stretchy regions which may move with user's garment better. Filaments are woven onto layers of nonwoven fabric layers in non-stretched state as described in detail in reference U.S. Pat. No. 8,574,211 hereby incorporated by reference. The mesh sheet 6 entails a layered grid of appropriate thermoplastic elastomer filaments. Materials would be blends of thermoplastic elastomer.

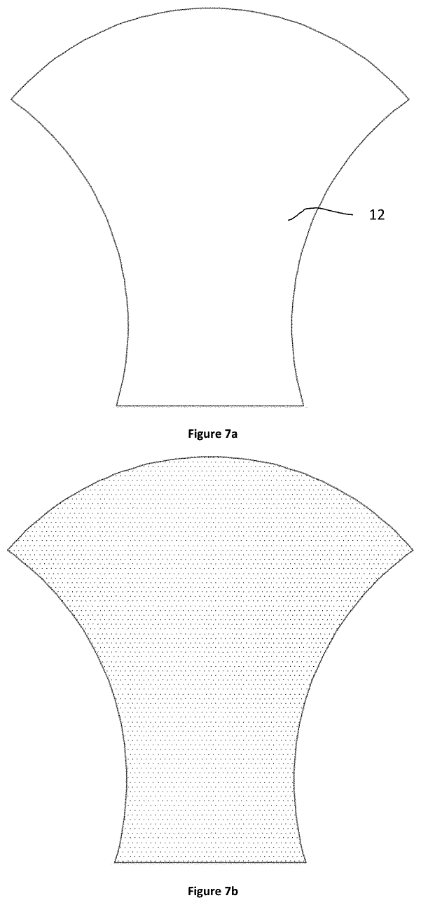

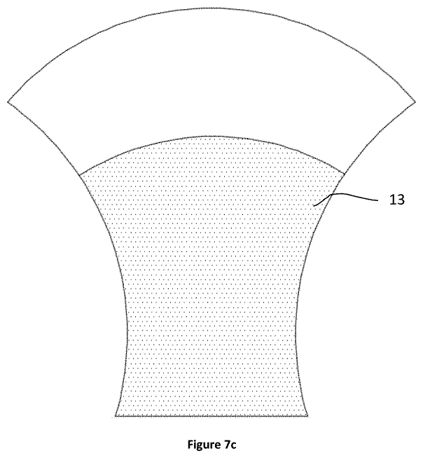

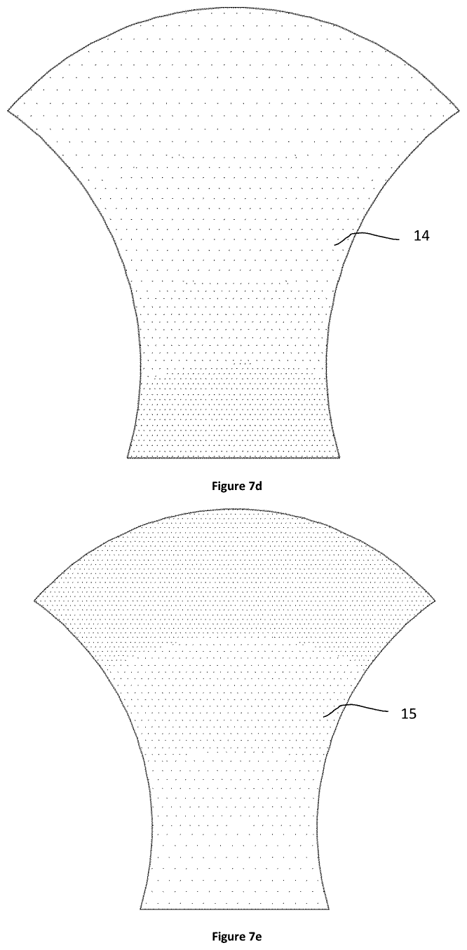

The back-side or garment-facing sheet 3 is substantially the same as skin contact sheet 4 with some differences. In one example, the sheet would also have perforations (as shown in FIG. 7b) much like the skin-contact sheet to allow flow of liquids. In another liquid impermeable embodiment, the back-side sheet 3 would not have any perforations and comprise laminate sheet(s) 12 made up of polyethylene and polypropylene or alternatively a non-woven fabric chemically treated to be hydrophobic (FIG. 7a). The back-side sheet 3 could also have a sub-area 13 (FIG. 7c) which is perforated leaving other areas non-perforated. In another embodiment (FIG. 7d), the perforations can be variable in spacing 14 from tightly spaced such in areas where liquid flow is expected to broadly spaced such as in areas where flow is not expected to take place. Alternately, the spacing could also be broad where flow is high 15 while tightly spaced where it is low (FIG. 7e). In such case, flow of liquid would be directed outward to increase area exposure of the absorbent sheet 2 to maximize absorption of escaping wetness.

A peel-away sheet is made up of waxed or hydrophobic paper or other material typically used in the art for disposable sanitary napkins. The sheet is pulled away to expose an adhesive layer so wearer may affix the article to underwear.

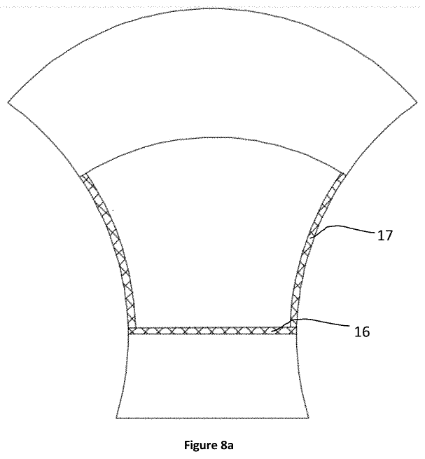

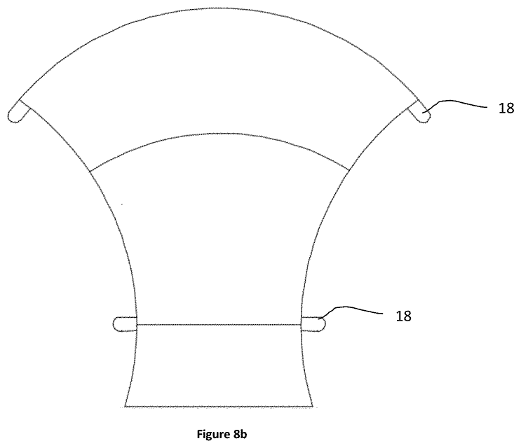

Disposed between the backside sheet 3 and the pull-away layer is a light adhesive as described in prior art such as in U.S. Pat. Nos. 8,029,483, 8,211,074 and related art. In the present invention, the adhesive could be applied in zones to attach the article firmly to the garment. Preferably, adhesive would be applied “Edge to edge” on the pantiliner. Alternatively, adhesives could be in a pattern in order to move with user and garment better or use of two or more different adhesives for differential stretching on surface of garment. Still another pattern of adhesives would entail adhesive strips only on particular spots or on a strip around the perimeter of the article. The article may also have index or indicator markings in order to help the user or caregiver properly attached the article to the undergarment. FIG. 8a shows markings 16 where a user should align the narrowest part of the undergarment with pantiliner. Indicators 17 also show the user where to align the liner to the border of the undergarment. Alternatively, folding tabs or flaps 18 may be used to index or align the article to affix it to the undergarment (FIG. 8b).

The article assembly would be either folded into a square or rectangle to be wrapped in individual wrappers or rolled like a scroll into a cylindrical shape to be packaged in individual wrappers. Alternatively, they may be left unwrapped and placed in a container. As with many pantiliners, assemblies may also be stacked flat (unfolded or rolled) unwrapped and placed in an appropriately sized box.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 mm” is intended to mean “about 10 mm”.

All documents cited in the Detailed Description of the Invention are, in relevant part, incorporated by reference; the citation of any document is not to be construed as an admission that it is prior art with respect to the present invention.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

I claim:

1. A disposable protective article for undergarments comprising a thin absorbent sheet which intervenes between a liquid-permeable backside sheet and a liquid-permeable skin-contact surface sheet.

2. The article of claim 1 wherein thin absorbent member which intervenes between a liquid-impermeable backside sheet and a liquid-permeable skin-contact surface sheet

3. The article of claim 1 wherein a hydrophilic second sheet (also referred to as a transfer sheet) is disposed between the skin-contact surface sheet and the thin absorbent member.

4. The article of claim 1 wherein article has a liquid permeable mesh layer disposed between the thin absorbent layer and the backside-sheet.

5. The article of claim 1 wherein skin contact surface sheet comprises layers of materials such as nonwoven fabric of olefin such as polyester, or polyamide series or may also be regenerated fibers like rayon and cupra, or natural fibers, such as cotton.

6. The article of claim 1 wherein skin contact surface sheet comprises two to three layers embossed in a regular pattern with small perforations hole-size minimum of 0.3 mm.

7. The article of claim 1 wherein skin contact surface sheet comprises a basis weight of approximately 30 to 60 gsm and Total thickness is preferably 5 mm or less, most preferably 2 mm thick or less.

8. The article of claim 1 wherein skin contact surface sheet includes protrusions along article's border or in a pattern to retain flow of exudate within the confines of the article.

9. The article of claim 1 wherein skin contact surface sheet includes “velcro” like structure whole or in a pattern.

10. The article of claim 1 wherein the thin absorbent sheet comprises any one of following materials: cellulose, rayon, acetate fibers, acrylic acid or acrylic salt-based materials, or combinations thereof spread evenly to approximately 0.5 to 2 mm thick.

11. The article of claim 1 wherein the thin absorbent sheet comprises materials unevenly spread to comprise thicker sheet approximately where urine-exudate would flow and less thick in other areas.

12. The article of claim 1 wherein the thin absorbent sheet comprises terminates onto a sub-area of the article.

13. The article of claim 1 wherein the thin absorbent sheet comprises materials containing water-absorbing resin at less than 10% of composition.

14. The article of claim 1 wherein the thin absorbent sheet comprises basis weight of approximately 20 to 100 gsm, preferably 20 to 50 gsm and thickness of absorbent sheet would be less than 5 mm, preferably less than 2mm thick.

15. The article of claim 1 wherein the mesh sheet comprises a layered grid of appropriate thermoplastic elastomer or other stretchable filaments.

16. The article of claim 1 wherein the mesh sheet comprises a layered grid with grid hole size ranges from 1 mm to 20 mm, preferable from 5 mm to 10 mm.

17. The article of claim 1 wherein the mesh sheet comprises a layered grid with orientation is generally diagonally disposed relative to the article orientation but may comprise any orientation.

18. The article of claim 1 wherein the mesh sheet comprises a layered grid of variable grid size across the sheet.

19. The article of claim 1 wherein the back-side or garment-facing sheet comprises a sheet substantially the same as skin-contact sheet including perforations to allow flow of liquids.

20. The article of claim 1 wherein the back-side or garment-facing sheet comprises a perforated sub-area leaving other areas non-perforated.

21. The article of claim 1 wherein the back-side or garment-facing sheet comprises a perforated area variable in spacing from tightly spaced to broadly spaced.
